(19) United States
(12) Patent Application Publication
(10) Pub. No.: US 2024/0417786 A1
(43) Pub. Date: Dec. 19, 2024

(54) HIGH STOKES SHIFT FLUORESCENCE DYES FOR MULTIPLEX DETECTION

(71) Applicant: QIAGEN GMBH, Hilden (DE)

(72) Inventors: KEVIN MATTHÄI, Langenfeld (DE); CELIA FRANCOIS, Köln (DE)

(21) Appl. No.: 18/702,656

(22) PCT Filed: Oct. 20, 2022
(86) PCT No.: PCT/EP2022/079237

§ 371 Date: Apr. 18, 2024

(30) Foreign Application Priority Data
Oct. 20, 2021 (EP) 21203740.2

(51) Int. Cl. C12Q 1/6853; C12Q 1/686

(52) U.S. Cl. CPC C12Q 1/6853; C12Q 1/686

(57) ABSTRACT

The invention relates to the use of a compound, wherein the compound is a fluorescence dye with a high Stokes shift, which is large enough to skip at least one intermediate emission filter channel, i.e., to jump at least into the next but one channel, as reference dye for non-droplet based dPCR systems to evaluate filling levels of compartments, as reference dye for fluorescence PCR systems for normalization or as dye for target detection for multiplex PCR, wherein the compound is bound to a probe or primer as fluorescent label, wherein the PCR is selected from the group consisting of digital PCR (dPCR), quantitative PCR (qPCR), reverse transcription PCR (RT-PCR), reverse transcription quantitative PCR (RT-qPCR) and real-time PCR.

### FIELD OF THE INVENTION

The present invention relates to the field of molecular biology, more particularly to the quantification of nucleic acid molecules in a sample.

### BACKGROUND OF THE INVENTION

Digital polymerase chain reaction (digital PCR, DigitalPCR, dPCR, droplet-digital PCR (ddPCR), or dePCR) is a biotechnological refinement of conventional polymerase chain reaction methods that can be used to directly quantify and clonally amplify nucleic acids strands including DNA, cDNA or RNA. The key difference between dPCR and traditional PCR lies in the method of measuring nucleic acids amounts, with the former being a more precise method than PCR, though also more prone to error in the hands of inexperienced users.

A “digital” measurement quantitatively and discretely measures a certain variable, whereas an “analog” measurement extrapolates certain measurements based on measured patterns. Traditional PCR carries out one reaction per single sample. dPCR carries out a single reaction within a reaction mixture physically separated into many partitions such that each partition contains ideally one target molecule at most. After multiple PCR amplification cycles, the partitions are checked for amplification with a binary readout of “0” or “1”. This separation allows a more reliable collection and sensitive measurement of nucleic acid amounts. The method has been demonstrated as useful for studying variations in gene sequences—such as copy number variants, point mutations or gene expression studies—and it is routinely used for clonal amplification of samples for next-generation sequencing.

Digital PCR (dPCR) uses the procedure of end-point PCR but splits the PCR reaction into many single partitions, in which the template is randomly distributed across all available partitions. After PCR, the amplification target is detected by measuring the increase in fluorescence signal—of e.g., either sequence-specific DNA/cDNA probes or intercalating dyes—in all positive partitions. As the template is distributed randomly, Poisson statistics can be used to calculate the average amount of target DNA per valid, analyzable partition. The total amount of target DNA in all partitions of a well is calculated by multiplying the amount of average target DNA per partition with the number of valid partitions. Calculation of target concentration is determined by referring to the volume in all analyzable partitions, that is, partitions which were filled with reaction mix. The total number of filled partitions at least for chip/plate based systems is identified by a fluorescent reference dye present in the reaction mix itself. Absolute quantification by dPCR eliminates the need for standard curves to determine amounts of target DNA in a given sample.

Hydrolysis probes, also known as TaqMan probes, are a popular detection chemistry for monitoring sequence-specific amplification in qPCR or digital PCR (dPCR). Just like with SYBR Green dye, signal detection is achieved through monitoring an increase in fluorescence as the reaction proceeds. But, the fluorescent signal in TaqMan™ chemistry is dependent on probe hydrolysis, rather than hybridization, hence the name “hydrolysis probes”. In the hydrolysis probes set up, there are two primers and a probe. The probe is also designed complementary to the target and contains a fluorophore and a quencher on either end. The TaqMan method allows using multiple fluorescence report dyes, such as FAM, VIC, NED, 5- and/or 6-carboxy-X-rhodamine (e.g., available commercially as ROX™ and as SuperROX™ (from Biosearch Technologies, Petaluma, Calif.)), and CY5, each of which have different emission spectra for detection and measurement of multiple target DNA sequence amplifications in a single reaction (multiplex PCR).

During the amplification process, the probe binds to the specific target sequence during the annealing step. The fluorophore is at the 5′ end of the probe, and the quencher moiety is usually located at the 3′ end or internally. Because of the proximity between the donor (fluorophore) and acceptor (quencher) on the probe, there is nearly no fluorescence. During the extension step, the 5′-3′ exo activity of the polymerase, hydrolyses the probe relieving the fluorophore from quenching effects and fluorescence can be detected and quantified in digital PCR. The fluorescence is proportional to the amount of accumulated PCR product.

Digital PCR provides certain advantages over other commonly used DNA quantification strategies (e.g. QIAxcel and qPCR). With absolute quantification of single molecules digital PCR uses less amounts of input DNA and does not require the back-calculation of the library against an average size determined by a Bioanalyzer assay. This makes dPCR-based quantification less time- and reagent-consuming while still providing similar sensitivity and accuracy as qPCR. Also it is more sensitive compared to quantifications using QuBit and PicoGreen (Table 1). Another advantage of digital PCR is that amplification occurs in separate partitions. Even if amplification efficiency differs from amplicon to amplicon or extraction to extraction, sufficient amplicon will be generated in a dPCR run to determine whether a target was present. Digital PCR will therefore provide binary values (present, even if poorly amplified; or absent) for the template of interest, that, corrected for the possibility of having one or more template molecules per partition using Poisson statistics, reveals absolute quantification of molecules/p.

TABLE 1
Summary of NGS library quantification methods.

| Method/ | Requires | | |
|---|---|---|---|
| Instrument | Bioanalyzer? | Quantification | Sensitivity |
| QIAxcel | Yes | Estimation through average library | pg-ng |
| | | molecular size | |
| PicoGreen | Yes | Estimation through average library | pg-ng |
| | | molecular size | |
| qPCR | Yes | Estimation through average library | 0.1-1 pg |
| | | molecular size | |
| dPCR | No | Absolute quantification | fM |
| | | of molecules per μl | |

Droplet digital PCR (ddPCR), also referred to as emulsion PCR (ePCR), is the most common kind of dPCR. In ddPCR the PCR solution is divided into smaller reactions through a water oil emulsion technique, which are then made to run PCR individually. The PCR sample is partitioned into nanoliter-size samples and encapsulated into oil droplets. The oil droplets are made using a droplet generator that applies a vacuum to each of the wells. Approximately 20,000 oil droplets are made from each 20 μL sample. In ddPCR the aqueous phase comprises one or more species of a polynucleotide templates, beads, enzymes, salts, buffers, and oligonucleotide primers, for amplifying said template. ddPCR is useful for studying variations in gene sequences, such as copy number variants and point mutations and is used for molecular diagnostics applications.

Other kinds of dPCR technologies known in the art use microwell plates or chips (e.g. QIAcuity technology, also called chip-based dPCR), capillaries, and arrays of miniaturized chambers with nucleic acid binding surfaces to physically partition the reaction mixture or sample, respectively.

Compared to droplet digital PCR (ddPCR), as provided by BioRad, dPCR on the QIAcuity system uses 96 and 24 well nanoplates in standard format (SBS format) that are suitable for usage in fully automated workflows, for e.g. reaction mix setup by QIAgility.

To perform dPCR PCR reactions are typically prepared on 24 or 96 well PCR plates, this input volume from each well is then transferred to further plates with a multitude of partitions which are connected by microchannels. After transfer of the sample volumes the connecting channels are closed to perform the PCR reaction. In the first step, the plate's microchannels and partitions are filled with the input volume in the wells. This is done by plunging of 24/96 pins in the elastic top seal and the input wells. This creates overpressure that pumps the input well liquid into the microchannels and partitions. Subsequentially, the connecting channels between the partitions are closed by a force controlled rolling process.

The second step is a high-accuracy plate thermocycler that performs the polymerase chain reaction. Several Peltier elements are used for the temperature generation and control.

The final step is the image acquisition of all wells. The partitions that have a target molecule inside emit fluorescence light and are brighter than those without target.

The optical system of the QIAcuity is a camera-based fluorescence microscopy system. The excitation source for the fluorescence dyes is a high-power white LED. This source in combination with a specific excitation filter is used to illuminate a whole well at a time. The fluorophores in the single partitions absorb that light and emit light that is being filtered by a detection filter, collected and imaged through an objective lens on a CMOS-camera chip. Depending on the configuration of the instrument (2plex/5plex), there are up to 2 or 5 selectable detection channels. An additional channel is used for detecting the base fluorescence of the reference dye of the master mix, to determine the exact number of filled partitions and normalization of fluorescence data. This, however, means that this channel cannot also be used for sample detection.

TABLE 2
Available channels in QIAcuity

| Channel | Excitation (nm) | Emission (nm) | Example fluorophores |
|---|---|---|---|
| Green | 463-503 | 518-548 | FAM ™, EvoGreen ® |
| Yellow | 514-535 | 550-564 | HEX ™, VIC ®, JOE ™ |
| Orange | 543-565 | 580-606 | TAMRA ™ |
| Red | 570-596 | 611-653 | ROX ™, Texas Red ® |
| Crimson | 590-640 | 654-692 | Cy5 ® |

In dPCR, the PCR reaction is separated into thousands of single partitions, in which the target molecules are randomly distributed across all available partitions. Some partitions will contain no copy of the target molecule, some will contain one copy of the target molecule, and some others will contain more than one copy of the target molecule. As the target molecules are distributed randomly, Poisson distribution can be used to calculate the copies of the target molecule per positive partition. The Poisson distribution gives probabilities for positive integer random events. The parameter of this distribution, A, is the expectation value for these events, which means it is the mean probability for a proportion of a counting process or the counting process for the dPCR analysis.

Published reference US patent application—US20150104797A1—applies a high Stokes shift dye as reference dye. The fluorescence dye with high Stokes shift is used in addition to standard passive reference dye (ROX) and does not replace the standard reference dye by a fluorescence dye with high Stokes shift.

### BRIEF DESCRIPTION OF THE INVENTION

In current PCR application fluorescence dyes are used to monitor and detect the PCR product (so called amplicon) creation during the thermal cycling process. Depending on the method the signal amplitude is used to detect amount of product build (qPCR), or within (d)dPCR the signal of the fluorescence dye is used to discriminate between compartments/droplets where product was created (positives) and compartments/droplets where no template was present for amplification/product generation (negatives). To detect PCR product generation such systems have specific filters to excite the fluorescence dye using a light source and a detection module with specific filters to detect the emitted light by the fluorescence dye. The filters narrow the light consisting of a mixture of wavelengths from ˜400 nm to ˜800 nm to a specific wavelength range allowing to excite and detect the one fluorescence dye but not the one which uses different wavelengths for excitation/emission (see FIG. 1).

Using narrow bandwidth filters shall limit the so-called cross talk (XT)/cross speech. This means that such fluorescence dye is predominantly measurable in one channel but not the other channel to prevent false positive signals.

Examples of such filter pairs and respective channels and possible fluorescence dyes (fluorophores) are listed in Table 2 and represent the equipment of QIAcuity device.

For non-droplet (e.g. nanoplate or chip) based dPCR systems a specific fluorescence dye (so called reference dye), which is present even without amplification, is used in one channel to monitor the filling results of the compartments. Hence this channel cannot be used for detection of the targets/PCR product generation. Due to the cross talk between fluorescence dyes and the reference dye specific channel the amount of channels for detection of different targets at once (so called multiplexing) is limited.

For example, the usual fluorescence dye FAM (IDT) has the excitation maxima at 495 nm and emission maxima at 520 nm (FIG. 2). As shown for such usual fluorescence dyes the maxima for excitation and emission are quite close to each other and the curves have a certain overlap. This shift between wavelength for excitation and wavelength for emission is called Stokes shift. To limit cross talk (XT) to neighbor channels the excitation and emission filter need to have a rather narrow bandwidth.

To increase the capability of multiplexing (more targets can be used within one reaction/sample vessel) or to use current dedicated reference channel for target detection the present invention uses a fluorescence dye with significantly higher Stokes shift (excitation maxima and emission maxima are much further away) than usual reference dyes or in another embodiment of the invention fluorescence dyes with high Stokes shift are used as target detection dyes to increase multiplexing capacity.

Using such kind of high Stokes shift fluorescence dyes (see FIGS. 3-5), the existing filter combinations are rearranged compared to filter combination used for usual fluorescence dyes to excite the high Stokes shift fluorescence dyes and detect their emitted light, respectively.

For example with the Atto490LS dye the green filter can be used for excitation and the crimson filter can be used for detection of emitted light. With such high Stokes shift the dye does not lead to interference to usual green or red/crimson dyes and can independently of PCR be used as reference dye to monitor the filling of the nanoplate/chip.

Such reference dye with high Stokes shift allows to use the current dedicated reference channel as target detection channel to increase the multiplexing capacity of the system. Therefore, the existing QIAcuity dPCR system for example could become a 6-plex system if the 6th channel is not used as reference channel.

Potential dyes for 6^{th} channel fitting to current filter of QIAcuity System would be, e.g., Alexa Fluor 680, Cy5.5, Quasar 705, ATTO 680, Alexa Fluor 700, Tye 705.

### BRIEF DESCRIPTION OF THE FIGURES

FIG. 1 shows transmission spectrum of example filter (Semrock, FF01-520/35); GMBW: guaranteed minimal bandwidth, FWHM: full width at half maximum.

FIG. 2 shows common excitation/emission curves for usual fluorescence dyes as represented by example dye: FAM (IDT).

FIG. 3 shows excitation/emission curves for 1^{st} example fluorescence dye with high Stokes shift: abberior STAR 520SXP.

FIG. 4 shows excitation/emission curves for 2^{nd} example fluorescence dye with high Stokes shift: Chromeo™ 494

FIG. 5 shows excitation/emission curves for 3^{rd} example fluorescence dye with high Stokes shift: ATTO 490LS Azide.

FIG. 6 shows an overview of PCR plate fluorescence reading. It can be observed that valid partitions (highlighted in blue) could be found and no major issue could be seen as the valid partitions are uniformly distributed across a well.

FIG. 7 shows 1D scatterplots from QIAcuity Software suite at 0.2 μM/0.5 μM ATTO490LS. At 0.5 μM ATTO680LS yield a relative fluorescence (RFU) of about 95 (FIG. 7B), this is similar compared to current reference dye with RFU of about 80 (FIG. 7A), at 0.2 μM ATTO680LS yield a relative fluorescence (RFU) of about 40 (FIG. 7C).

FIG. 8 shows as an example 1D scatterplots from green channel (FIG. 8A) and 1D scatterplot from crimson channel (FIG. 8B) at 0.5 μM of ATTO490LS. The observed fluorescence signals do not indicate crosstalk between the channels.

FIG. 9 shows exemplary 1D scatterplot for all fluorescence channels from a 5-plex reaction at 0.5 μM of ATTO490LS.

FIG. 10 indicate that the overall observed concentration is within the expected range of +/−10% on average for all used channels, data shown were obtained in experiments with 0.5 μM of ATTO490LS.

FIG. 11 shows exemplary 1D scatterplot for target detection via PCR with ATTO490LS or Chromeo 494. A: Target channel of NTC (non-template control) and three positive wells from assay QNIC using ATTO490LS probe. B: Target channel of NTC (non-template control) and three positive wells from assay ERBB2 using ATTO490LS probe. C: Target channel of NTC (non-template control) and three positive wells from assay QNIC using Chromeo494 probe. D: Target channel of NTC (non-template control) and three positive wells from assay ERBB2 using Chromeo494 probe.

FIG. 12 shows exemplary 1D scatterplot from 3-plex reaction from target detection with probes labelled with FAM, Cy5 and ATTO490LS or Chromeo 494.

FIG. 13 shows reference channel 1D scatterplot and target channel (ERBB2 in FAM—1D scatterplot from example 4). A: Target channel of NTC (non-template control) and three positive wells (D6-D8) from assay ERBB2 FAM. B: Reference channel of three positive wells from assay ERBB2 FAM (Part A) using Chromeo 494 dye bound to some nucleotides (fragments from polymerase digestion of QNIC probe labelled with Chromeo 494). C: Target channel of NTC (non-template control) and three positive wells (D6-D8) from assay ERBB2 FAM. D: Reference channel of three positive wells from assay ERBB2 FAM (Part A) using ATTO490LS bound to some nucleotides (fragments from polymerase digestion of QNIC probe labelled with ATTO490LS).

FIG. 14 shows reference channel 1D scatterplot using ATTO490LS dye two times imaged (Imaging 1 and Imaging 2).

FIG. 15 shows for three wells the 1D scatterplot for each 6 targets channel, used filter pairs and measured concentration respectively. As reference dye ATTO490LS was used (reference scatterplot not shown).

### DETAILED DESCRIPTION OF THE INVENTION

Analysis or examination herein refers to, identifying whether or not amplification has taken place, identifying whether or not the target sequence lies between the primer regions and optionally, has the right length, identifying the amount of amplification product with a correct target sequence. Preferably in the method of the invention precise quantification of the amplification product is desired.

Application of a fluorescence dye for target detection implies either use of intercalating dyes in a PCR reaction to detect formation of double-stranded DNA molecules, or labelling primers or probes with suitable fluorophore molecules to observe presence of formation of nucleic acid molecules with complementary target sequences in dependance on the detected fluorescence signals of the fluorophore molecules. By using different fluorophores with distinct emission and excitation spectra it is possible to combine more than one detection system into one PCR reaction (multiplex PCR) and to separately detect the fluorescence signals. Potential labelling molecules include but are not limited to fluorescence dye or chemiluminescence dye in particular a dye of the cyanine type. In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like. In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

“Digital quantification” as used herein refers to a process that quantifies nucleic acids copy numbers without the use of standard curves by means of a digital amplification method, such as digital PCR (dPCR). Therefore, “digital quantification” allows an absolute quantification of nucleic acids copy number.

“Sample molecules” or “individual sample templates” as used herein refers to any kind of nucleic acid molecules contained in a sample to by analyzed which can be subjected to dPCR, such as single-stranded or double-stranded DNA and/or RNA.

The present nucleic acid amplification product to be analyzed may stem from the amplification of genomic DNA, mitochondrial DNA, chloroplast DNA, cDNA, or the like, from any suitable source. The fragments (target) may have any suitable length, such as about 10 to 10,000, or 20 to 2,000 nucleotides, among others. The fragments may or may not be size-selected before attachment to the adapters (primer regions). Fragments may be generated from a source nucleic acid material by any suitable approach, such as shearing, chemical digestion, enzymatic digestion, amplification with one or more primers, reverse transcription, end-polishing, or any combination thereof, among others. The fragments may have flush or overhanging ends and may be at least predominantly double-stranded or single-stranded. The target nucleic acid may be RNA or DNA. DNA is preferred.

The “labelling” of the DNA or RNA strands can be realized by associating or incorporating any kind of marker detectable by conventional imaging techniques, e.g. a fluorescent marker.

“Reaction mixture” as used herein refers to the solution constituting the reaction environment. A reaction mixture according to the invention includes any solution known in the art allowing an amplification of nucleic acid molecules.

The “amplification” of the individual sample templates refers to any kind of nucleic acid amplification method which results in the generation of multiples of the original template.

The “quantification” as used herein can be realized by any kind of method allowing the counting of the individual DNA strands.

In one embodiment of the present invention a fluorescence dye is used with a high Stokes shift, which is large enough to skip at least one intermediate emission filter channel, i.e., to jump at least into the next but one channel. The high Stokes shift dyes of this invention have a Stokes shift of at least 60 nm, optionally at least 70 nm, optionally at least 80 nm, optionally at least 90 nm, optionally at least 100 nm, optionally at least 150 nm and optionally at least 200 nm. In this embodiment the high Stokes shift dye is used as reference dye for non-droplet based dPCR systems to evaluate filling levels of compartments, wherein positive detection of the fluorescence signal of the reference dye indicates that the respective compartment is filled with PCR MasterMix and thus should be included in the final analysis as either a positive or a negative sample depending on the detection of target specific fluorescence signal.

In another embodiment of the present invention a fluorescence dye is used with a high Stokes shift, which is large enough to skip at least one intermediate emission filter channel, i.e., to jump at least into the next but one channel. The high Stokes shift of the dyes of this invention have a Stokes shift of at least 60 nm, optionally at least 70 nm, optionally at least 80 nm, optionally at least 90 nm, optionally at least 100 nm, optionally at least 150 nm and optionally at least 200 nm. In this embodiment the high Stokes shift dye is used as reference dye for fluorescence PCR systems for normalization. Normalization reference dyes are applied in fluorescence PCR systems to adjust for potentially varying fill levels of individual PCR well.

In another embodiment of the present invention a fluorescence dye is used with a high Stokes shift, which is large enough to skip at least one intermediate emission filter channel, i.e., to jump at least into the next but one channel. The high Stokes shift of the dyes of this invention have a Stokes shift of at least 60 nm, optionally at least 70 nm, optionally at least 80 nm, optionally at least 90 nm, optionally at least 100 nm, optionally at least 150 nm and optionally at least 200 nm. In this embodiment the high Stokes shift dye is used as dye for target detection for multiplex PCR, wherein the compound is bound to a probe or primer as fluorescent label, wherein the PCR is selected from the group consisting of digital PCR (dPCR), quantitative PCR (qPCR), reverse transcription PCR (RT-PCR), reverse transcription quantitative PCR (RT-qPCR) and real-time PCR. Use of high Stokes shift fluorescence dyes for target detection allows for an increased degree of multiplexing as more potential combinations of excitation and emission filters can be specifically selected to detect a pre-defined fluorophore and thus an associated primer or probe which is used to selectively bind the target nucleic acid molecules.

The high Stokes shift dyes of this invention may be used as solubilized free molecules. Alternatively, the dyes may be bound to further non-dye elements such as but not limited to protective groups. In some embodiments the high Stokes shift dye is bound to oligonucleotides. Binding of the dyes to oligonucleotides may at least achieve better protection from degradation.

The use of high Stokes shift fluorescence dyes as reference dyes or for target detection in multiplex PCR reactions requires, that the selected high Stokes shift fluorescence dye does not interfere with use of regular fluorescence dyes for PCR products or reference dye for estimation of partition filling, allowing higher degree of multiplexing, wherein regular fluorescence dyes are characterized by a Stokes shift of less than 60 nm. This means that the combinations of excitation and emission filters and fluorescence dyes with their specific excitation and emissions spectra have to be selected in such a way to result in no or minimal cross-talk between the various fluorescence channels. The person skilled in the art is aware how to optimize this selection. Fluorescence filters with narrow transmission bandwidth and fluorescence dyes with narrow emission peaks are preferable for this application.

In another embodiment of the present invention the high Stokes shift fluorescence dye is used as a reference dye or for target detection to enable a higher degree of multiplexing, wherein the higher degree of multiplexing comprises an increase of the degree of multiplexing by at least 1, preferably by at least 2, more preferably by at least 3, even more preferably by at least 4, even more preferably by at least 5, even more preferably by at least 6, even more preferably by at least 7, even more preferably by at least 8, even more preferably by at least 9 and most preferably by at least 10.

In another embodiment of the present invention the high Stokes shift fluorescence dye is used as a reference dye or for target detection to enable a higher degree of multiplexing, where this may comprise detection of at least 3 PCR products in one PCR reaction, optionally of at least 4 PCR products in one PCR reaction, preferably at least 5 PCR products in one PCR reaction, more preferably at least 6 PCR products in one PCR reaction, even more preferably at least 7 PCR products in one PCR reaction, even more preferably at least 8 PCR products in one PCR reaction, even more preferably at least 9 PCR products in one PCR reaction, even more preferably at least 10 PCR products in one PCR reaction, even more preferably at least 11 PCR products in one PCR reaction and most preferably at least 12 PCR products in one PCR reaction.

High Stokes shift fluorescence dyes of the present invention may be selected from but are not limited to the group consisting of Alexa Fluor 680, Cy5.5, Quasar 705, ATTO 680, Alexa Fluor 700 and Tye 705.

In another embodiment of the present invention the high Stokes shift fluorescence dye is used for performing multiplex PCR on non-droplet based dPCR systems, nucleic acid amplification with multiplex reactions or multiplex PCR, wherein the PCR is selected from the group consisting of digital PCR (dPCR), quantitative PCR (qPCR), reverse transcription PCR (RT-PCR), reverse transcription quantitative PCR (RT-qPCR) and real-time PCR.

In another embodiment of the present invention the high Stokes shift fluorescence dye is used for target detection, wherein the dye is bound to a probe or primer as fluorescent label and is used for an application selected from the group consisting of absolute quantification of nucleic acids in a sample, mutation detection in nucleic acids, analysis of genome editing, analysis of copy number variation and analysis of gene expression.

In another aspect the present invention comprises a method for performing multiplex PCR on non-droplet based dPCR systems, nucleic acid amplification with multiplex reactions or multiplex PCR, wherein the PCR is selected from the group consisting of digital PCR (dPCR), quantitative PCR (qPCR), reverse transcription PCR (RT-PCR), reverse transcription quantitative PCR (RT-qPCR) and real-time PCR, wherein a high Stokes shift fluorescence dye is added to the PCR reaction as reference dye or dye for target detection, wherein the high Stokes shift dye is bound to a probe or primer as fluorescent label.

In another embodiment of the present invention the method for multiplex PCR is performed for an application selected from the group consisting of absolute quantification of nucleic acids in a sample, mutation detection in nucleic acids, analysis of genome editing, analysis of copy number variation and analysis of gene expression.

In another embodiment of the present invention high Stokes shift fluorescence dyes may be applied in methods for multiplex detection of biological or chemical components in a sample, wherein the high Stokes shift fluorescence dye is added as reference dye or dye for target detection.

In another aspect the present invention comprises the use of high Stokes fluorescent dyes in a composition as PCR mastermix, the composition comprising:
a) at least one fluorescent dye with a high Stokes shift, which is large enough to skip at least one intermediate emission filter channel, i.e., to jump at least into the next but one channel,
b) a heat-resistant DNA polymerase,
c) mixture of dNTPS,
d) a PCR buffer solution and
e) bivalent ions, preferably Mg2+ ions.

The invention also relates to a composition comprising:
a) at least one fluorescent dye with a high Stokes shift, which is large enough to skip at least one intermediate emission filter channel, i.e., to jump at least into the next but one channel,
b) a heat-resistant DNA polymerase,
c) mixture of dNTPS,
d) a PCR buffer solution and
e) bivalent ions, preferably Mg²⁺ ions.

A further aspect of the present invention is a kit for performing the method for multiplex PCR reactions. The features, characteristics, advantages and embodiments disclosed for the high Stokes fluorescent dye as well as the methods according to the invention apply likewise to the kit according to the invention.

A kit is a combination of individual elements useful for carrying out the method of the invention, wherein the elements are optimized for use together in the methods. The kit may also contain additional reagents, chemicals, buffers, reaction vials etc. which may be useful for carrying out the method according to the invention. Such a kit unifies all essential elements required to work the method according to the invention, thus minimizing the risk of errors. Therefore, such kits also allow semi-skilled laboratory staff to perform the method according to the invention.

The person skilled in the art is well aware that a kit for PCR reactions should contain at least the reagents for generating individual DNA strands, reagents for isothermal amplification based on the template DNA strands, including DNA polymerases; and/or reagents for enhancing an isothermal amplification; and/or reagents for incorporation detection marker into amplified DNA strands for detection and quantification; and/or reagents for stabilizing secondary structures of individual DNA strands.

The kit according to the invention comprises:
a) a composition comprising:
   a. at least one fluorescent dye with a high Stokes shift, which is large enough to skip at least one intermediate emission filter channel, i.e., to jump at least into the next but one channel,
   b. a heat-resistant DNA polymerase,
   c. mixture of dNTPS,
   d. a PCR buffer solution and
   e. bivalent ions, preferably Mg²⁺ ions;
b) a container and
c) a manual.

According to the invention, the above reagents include, but are not limited to, DNA polymerases with strong strand displacement activity such as phi 29 DNA polymerase, Bst DNA polymerase (large fragment), SensiPhi DNA polymerase, RNA polymerase; ssDNA and/or dsDNA ligases (e.g. CircLigase ssDNA ligase, T4 DNA ligase); reverse transcriptases; kinases such as T4 polynucleotide kinase; nicking enzymes; exonucleases such as TS exonuclease, T7 exonuclease; DNA polymerases with proof-reading activities (3′ to 5′ exonuclease activity) such as Phi29 DNA polymerase, T4 DNA polymerase, T7 DNA polymerase, Phusion DNA polymerase; buffers for afore-mentioned components.

It is to be understood that the before-mentioned features and those to be mentioned in the following cannot only be used in the combination indicated in the respective case, but also in other combinations or in an isolated manner without departing from the scope of the invention.

For the application of high Stoke shift dyes according to the invention the software of the applied PCR system needs to provide the user options to customize the filter settings (both excitation and emission filters) and the number of fluorescence detection channels. Thus, the user may select manually the pairings of excitation and emission filters for each channel as appropriate for the dye, which is to be detected in the respective channel, as well as the number of detection channels depending to the degree of multiplexing in the PCR reaction and also depending on the instruments available filter equipment.

### EXAMPLES

### Example 1

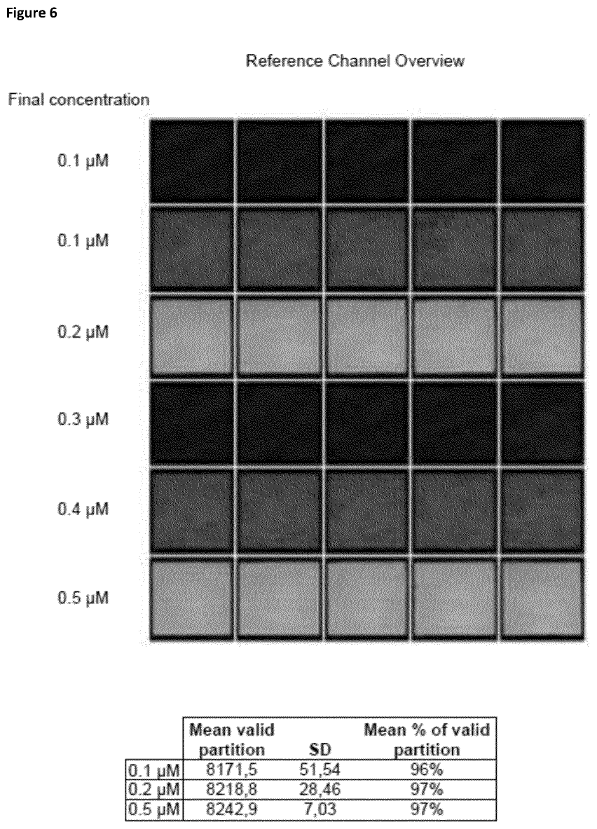

First test with ATTO490LS were performed by solubilizing the dye with water and using it as reference dye in QIAcuity system with concentrations of 0.2 μM to 0.5 μM. This test revealed that valid partitions, i.e., partitions filled with PCR MasterMix, could be found by image analysis. For this test a 96 well 8.5 k Nanoplate using QIAcuity Probe MasterMix was used and usual reference dye was replaced by ATTO490LS. No DNA template was added to see if filling is homogenous. To use ATTO490LS as reference dye the configuration of a QIAcuity instrument was altered and green filter used for excitation and crimson filter used for emission instead of usual reference filters. As result on average 97% of valid partition were found which is comparable to current reference dye. The overview in FIG. 6 shows that valid partitions (highlighted in blue) could be found and no major issue could be seen as the valid partitions are uniformly distributed across a well. Therefore no filling issue was observed and filling results are comparable to those using current reference dye.

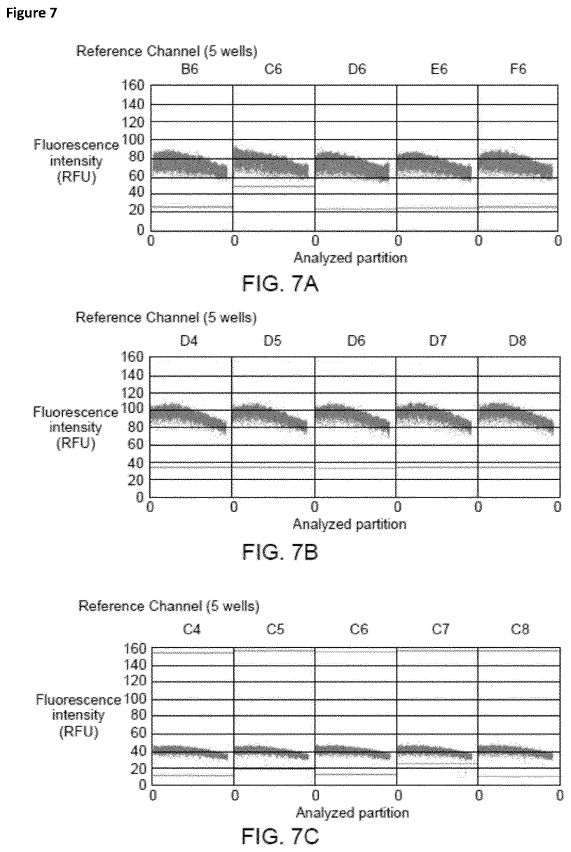

In addition FIG. 7 shows a 1D scatterplot from QIAcuity Software suite at 0.2 μM/0.5 μM ATTO490LS. At 0.5 μM ATTO680LS yield a relative fluorescence (RFU) of about 95 (FIG. 7B), this is similar compared to current reference dye with RFU of about 80 (FIG. 7A), at 0.2 μM ATTO680LS yield a relative fluorescence (RFU) of about 40 (FIG. 7C).

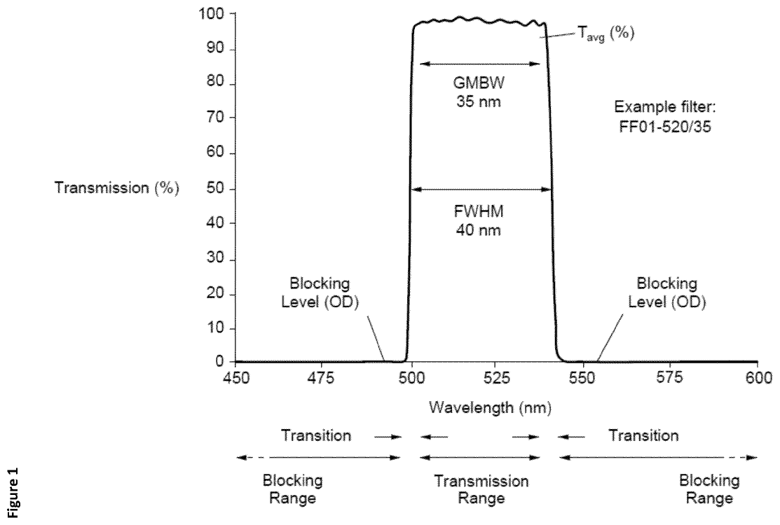

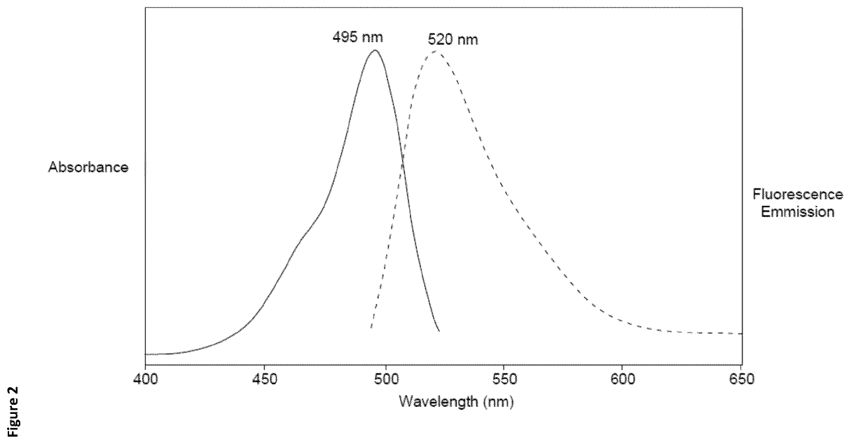

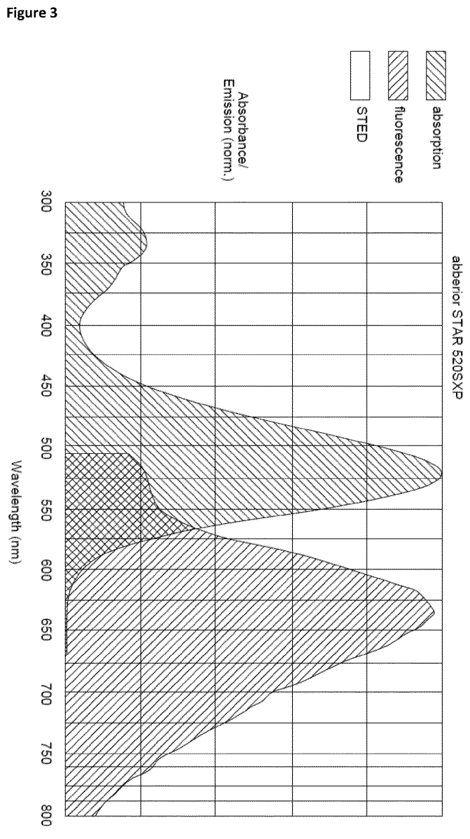

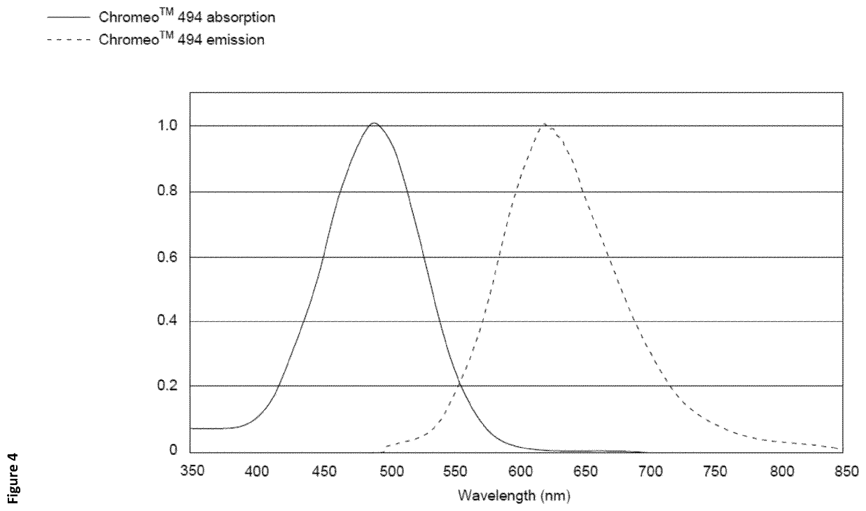

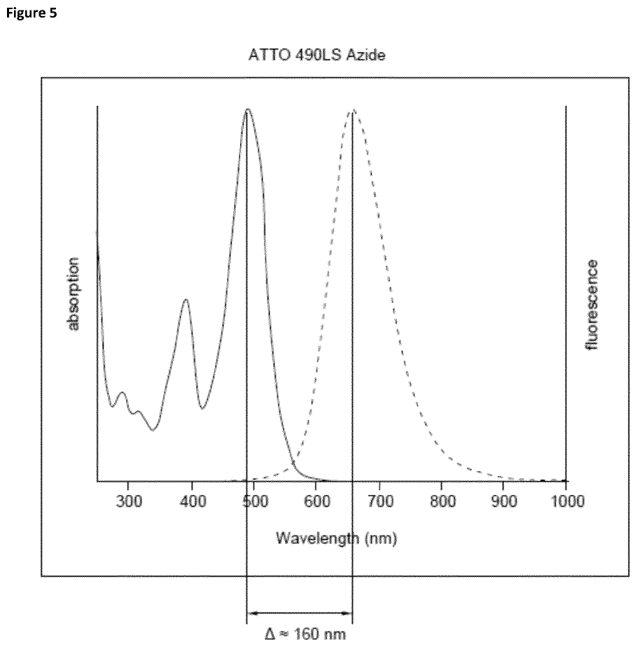

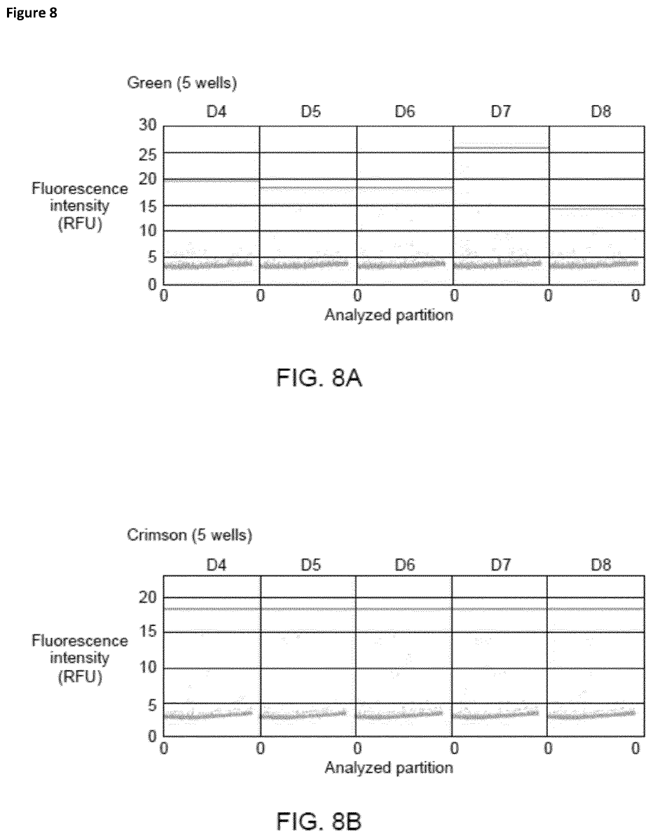

The same plate was also imaged in usual green and crimson channel to see of any so call cross talk is visible. No major cross talk can be observed. As example the 1D scatterplot from green channel (FIG. 8A) and 1D scatterplot from crimson (FIG. 8B) at 0.5 μM ATTO490LS is shown in FIG. 8.

### Example 2

In a second test a 96 well 8.5 k Nanoplate using QIAcuity Probe MasterMix was used and usual reference dye was replaced by ATTO490LS. DNA template of Quantinova IC in a concentration of 600 cp/μl was added to test if PCR reaction is influenced by presence of a different reference dye. To use ATTO490LS as reference dye the configuration of a QIAcuity instrument was altered and green filter used for excitation and crimson filter used for emission instead of usual reference filters. For this experiment 1-plex and 5-plex reactions and NTC wells (with no DNA template) were pipetted. As concentration for ATTO490LS dye 0.2 μM and 0.5 μM were used to see if different level of dye concentration create any PCR inhibitions. A usual PCR cycling of 2 min at 95° C. and 40×(15 s 95° C. and 30 s 60° C.) was performed, which is comparable to usual runs done on QIAcuity system.

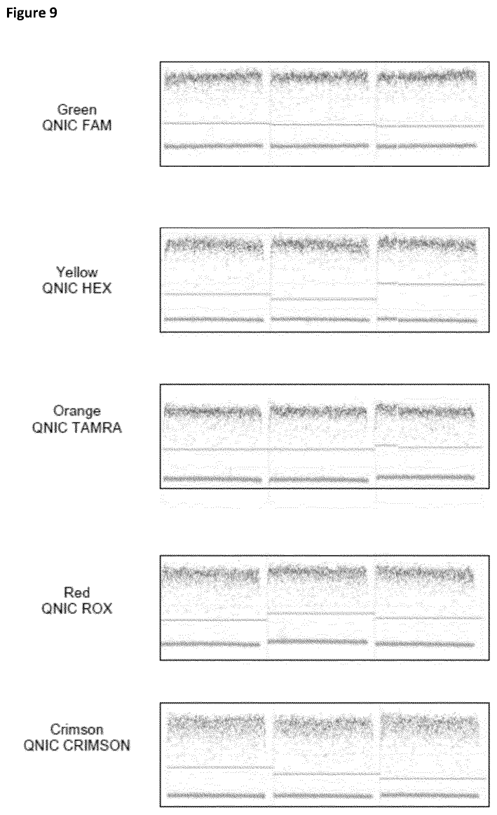

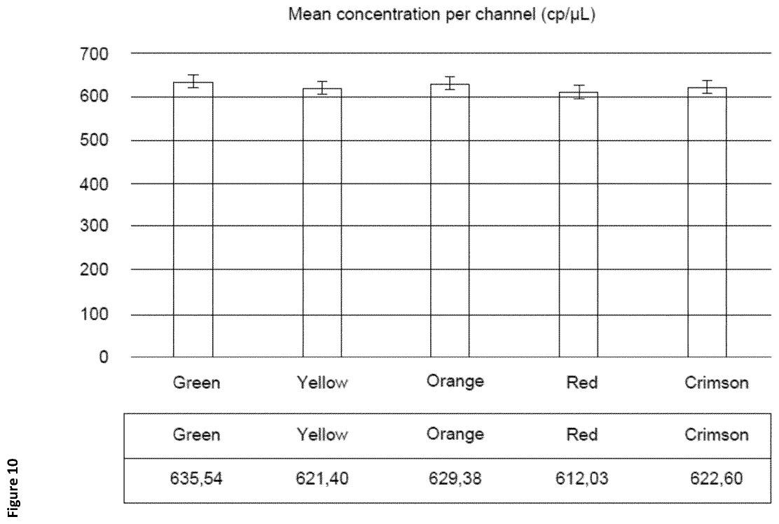

Neither at 0.2 μM nor at 0.5 μM a PCR inhibition could be detected and PCR results are comparable to usual PCR runs on a QIAcuity system. As example a 1D scatterplot for all fluorescence channels a from 5-plex reaction is shown in FIG. 9. The overall observed concentration is within the expected range of +/−10% on average for all used channels (FIG. 10).

Within NTC wells (wells with no DNA template) no side products (false positives) or increased amount of positives due to dust contamination were observed (data not shown). Overall the results are comparable to results usual obtained in similar experiments using the current reference dye.

### Example 3

In a third test a 96 well 8.5 k Nanoplate using QIAcuity Probe MasterMix was used with usual reference dye and high Stoke shift dye ATTO490LS and Chromeo 494 were used as dye attached to a PCR probe for target detection.

DNA template of Quantinova IC in a concentration of 800 cp/μl or gDNA for ERBB2 gene detection in a concentration of 500 cop/μL was added to test that high stoke dye can also be used for PCR amplification detection thus increasing the multiplex capability.

To be able to detect in target channel ATTO490LS and Chromeo 494 dye the configuration of a QIAcuity instrument was altered and green filter used for excitation and crimson filter used for emission instead of usual green filters.

For this experiment 1-plex reaction per probe and 3-plex reaction using 2 regular probes with normal Stoke shift and either an ATTO490LS or a Chromeo 494 dye labeled probe and NTC reactions (with no DNA template) were pipetted (NTC data not shown).

A usual PCR cycling of 2 min at 95° C. and 40×(15 s 95° C. and 30 s 60° C.) was performed, which is comparable to usual runs done on QIAcuity system.

Target amplification was detected for both assays QNIC and ERBB2 with both probes (ATTO490LS and Chromeo 494).

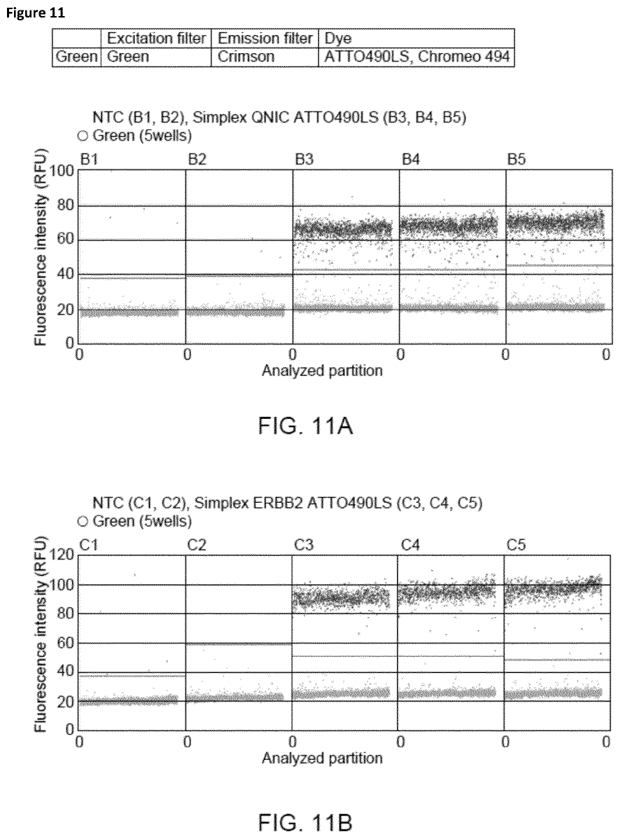

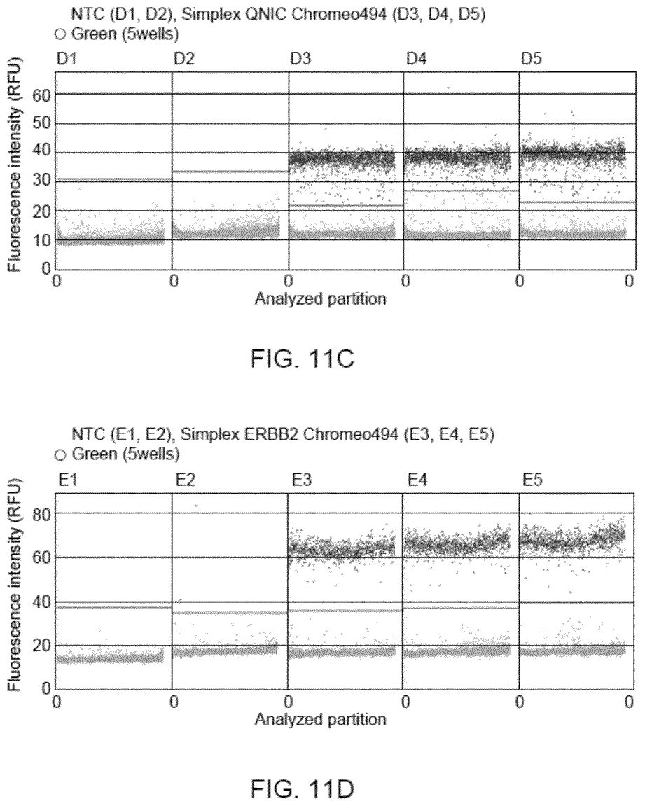

As example a 1D scatterplot for modified Green channels from 1-plex reaction is shown in FIG. 11.

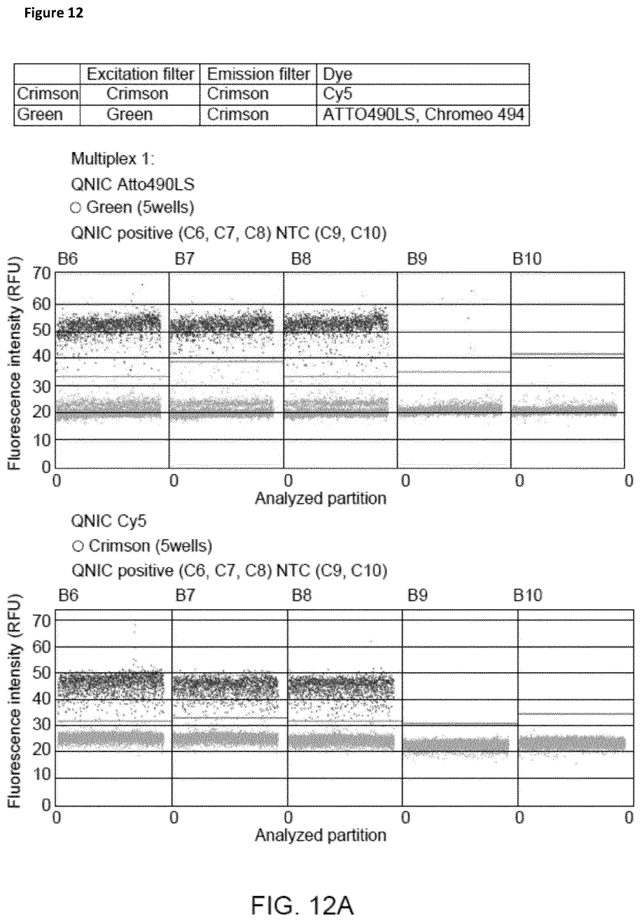

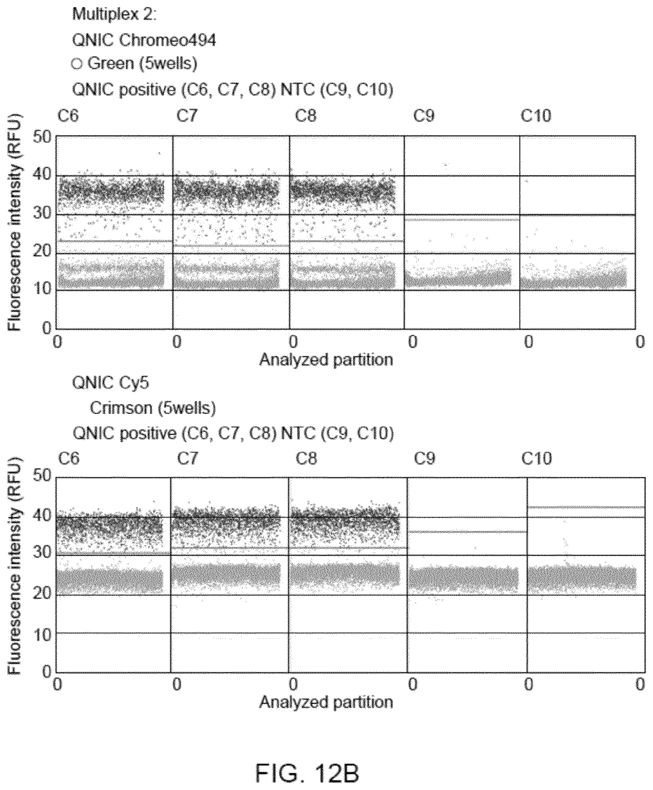

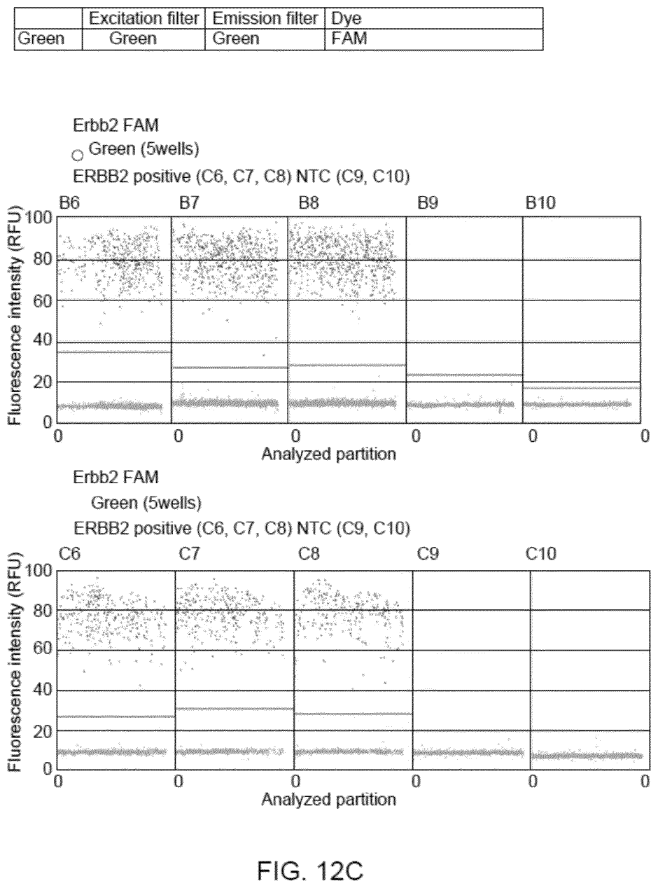

Moreover, 3-plex example in FIG. 12 shows that using high Stoke shift dye multiplexing capability can be increased. 1D scatterplot shows that a triplex can be performed with only 2 filters pair (here green and crimson—Target detected with Cy5 dye can be detected with standard Crimson filter pair. Target detected with FAM dye can be detected with standard Green filter pair, and finally target detected with ATTO 490LS or Chromeo 494 dye can be detected with Green and Crimson filter combination using in software green channel with modified filter settings).

### Example 4

In a fourth example, 96 well 8.5 k Nanoplate using QIAcuity Probe MasterMix was used but usual reference dye was replaced by ATTO490LS (compared to example 1 and 2 ATTO490LS dye was attached to some nucleotides similar to current reference dye to closely mimic the reference dye (usual reference dye is attached to 6 poly A nucleotide)) or Chromeo 494 (was attached to some nucleotides similar to current reference dye). For this example, DNA template of Quantinova IC was added at 20 000 cop/μL concentration to have only positive partition detected by high Stoke shift dye ATTO490LS or Chromeo 494. When the polymerase hydrolase activity digests the probe, the dye (here ATTO490LS or Chromeo 494) will be released with small amount of nucleotides simulating the current reference dye bound to 6×A (ployA tail). gDNA template at 600 cop/μL was added as standard amplification for ERBB2 gene detection with FAM dye.

To be able to detect in reference channel ATTO490LS and Chromeo 494 dye the configuration of a QIAcuity instrument was altered and green filter used for excitation and crimson filter used for emission instead of usual reference filters.

A usual PCR cycling of 2 min at 95° C. and 40× (15 s 95° C. and 30 s 60° C.) was performed, which is comparable to usual runs done on QIAcuity system.

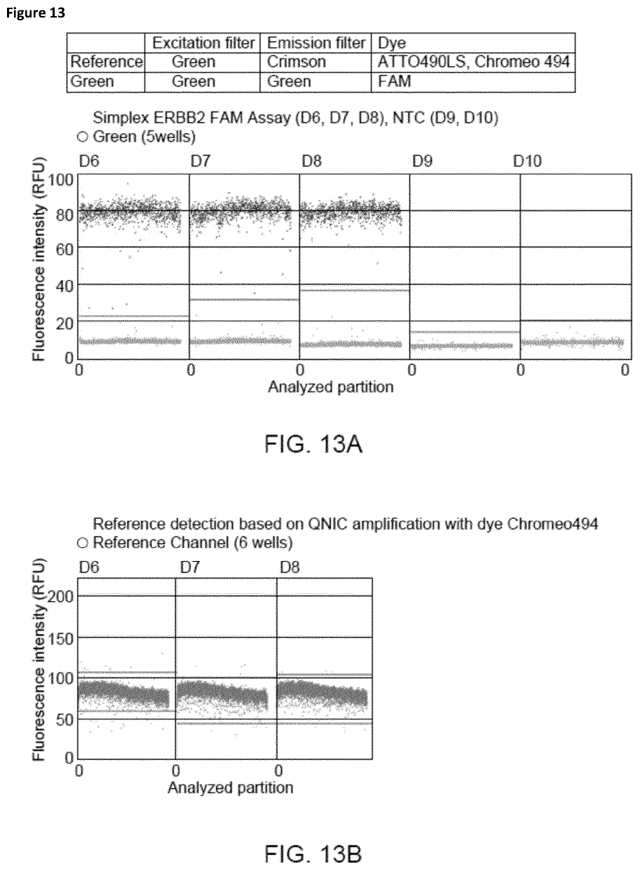

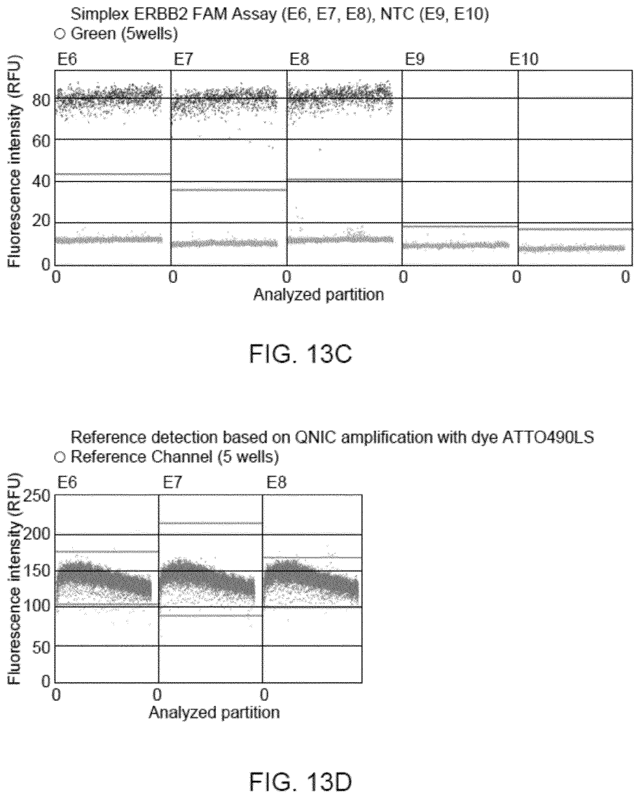

Results presented in FIG. 13 show that high Stoke shift dye ATTO490LS or Chromeo 494 signal were detected similar than using current reference dye. Standard amplification of interest (ERBB2 gene) was analyzable.

### Example 5

In a fifth example a 96 well 8.5 k Nanoplate using QIAcuity Probe MasterMix was used and usual reference dye was replaced by ATTO490LS. In addition 5 other regular probes/dyes were used to show using high stoke dye as reference can increase multiplexing capacity, here 6-plex reaction.

DNA template of Quantinova IC and gDNA was added as positive sample.

A usual PCR cycling of 2 min at 95° C. and 40×(15 s 95° C. and 30 s 60° C.) was performed, which is comparable to usual runs done on QIAcuity system.

To be able to detect in reference channel ATTO490LS and Chromeo 494 dye the configuration of a QIAcuity instrument was altered and green filter used for excitation and crimson filter used for emission instead of usual reference filters.

To be able to detect target amplification within reference filter pair, the configuration of a QIAcuity instrument was altered and regular crimson channel was altered using for crimson channel the reference filter pairs.

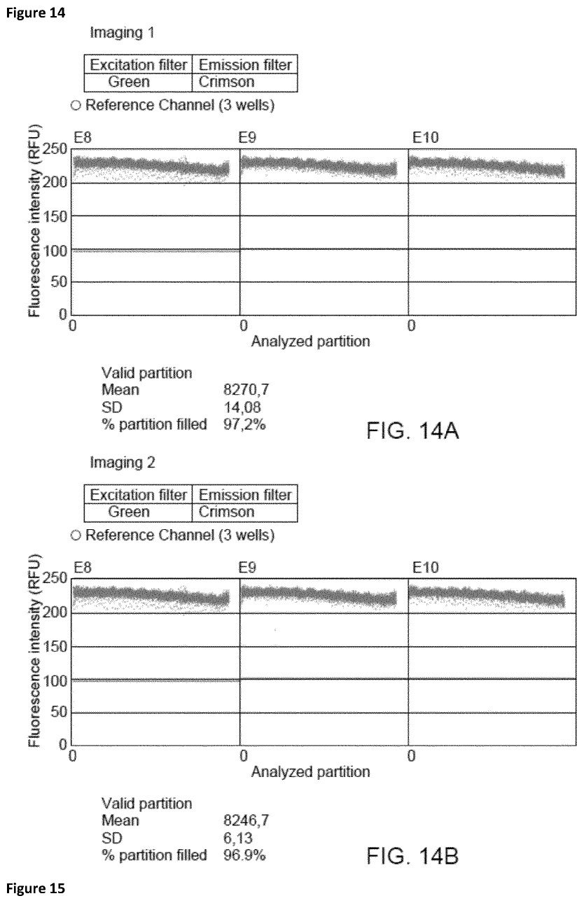

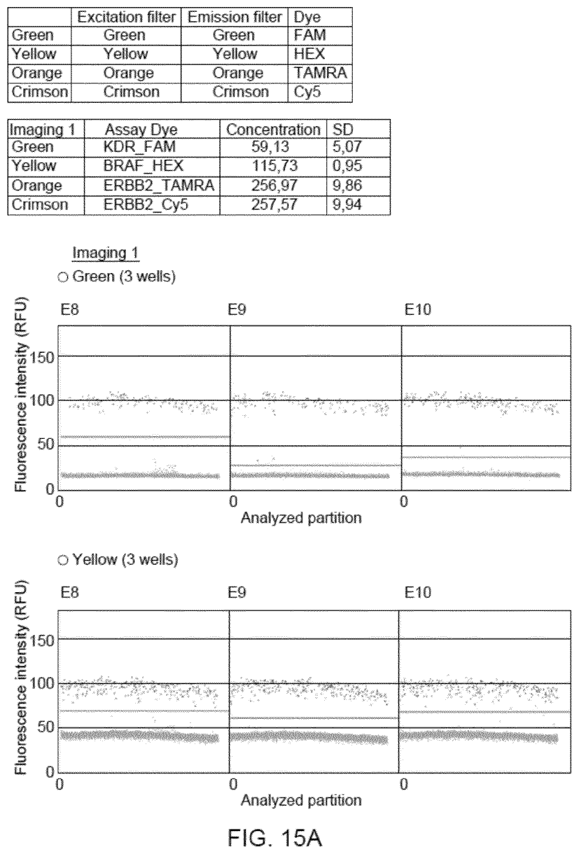

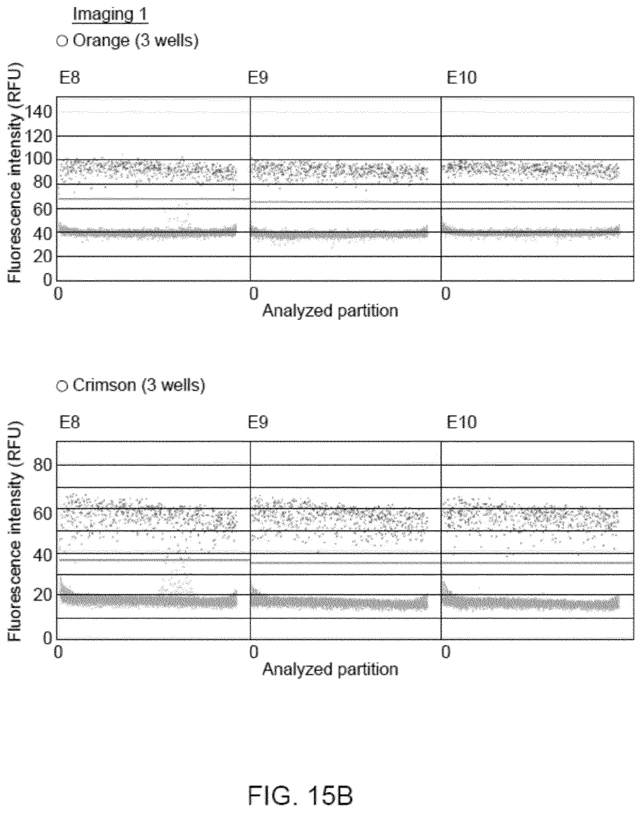

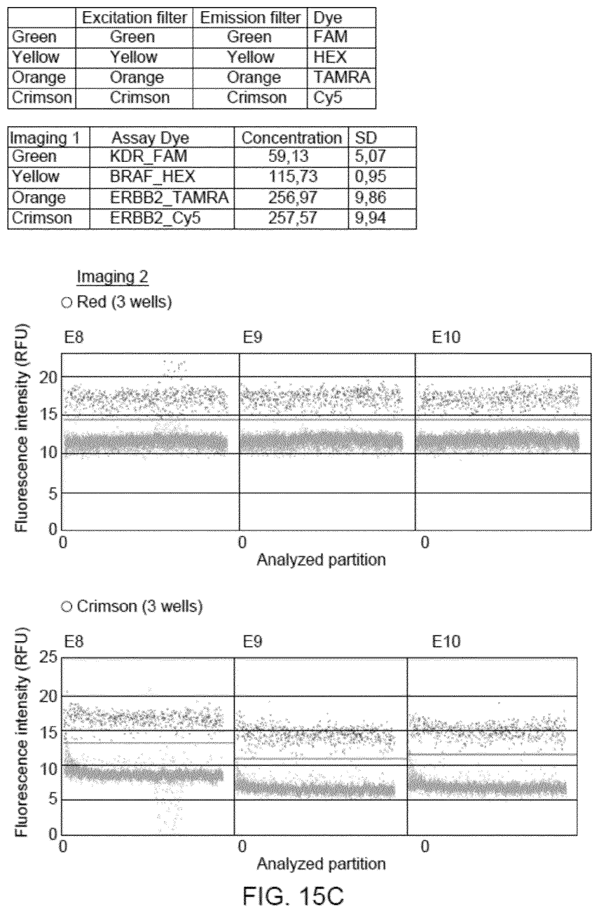

FIG. 14 shows that valid partition are detected using as reference dye ATTO490LS and FIG. 15 shows that all 6 targets were successfully detected.

1. Use of a compound, wherein the compound is a fluorescence dye with a high Stokes shift, wherein the high Stokes shift is at least large enough to skip at least one emission filter channel, as reference dye for non-droplet based dPCR systems to evaluate filling levels of compartments, as reference dye for fluorescence PCR systems for normalization or as dye for target detection for multiplex PCR, wherein the compound is bound to a probe or primer as fluorescent label, wherein the PCR is selected from the group consisting of digital PCR (dPCR), quantitative PCR (qPCR), reverse transcription PCR (RT-PCR), reverse transcription quantitative PCR (RT-qPCR) and real-time PCR.

2. Use of the compound of claim 1, where the high Stokes shift is at least 60 nm.

3. Use of the compound according to claim 1, wherein the compound does not interfere with use of regular fluorescence dyes for PCR products allowing higher degree of multiplexing, wherein regular fluorescence dyes are characterized by a Stokes shift of less than 60 nm.

4. Use of the compound according to claim 3, wherein the higher degree of multiplexing comprises an increase of the degree of multiplexing by at least 1, preferably by at least 2, more preferably by at least 3, even more preferably by at least 4, even more preferably by at least 5, even more preferably by at least 6, even more preferably by at least 7, even more preferably by at least 8, even more preferably by at least 9 and most preferably by at least 10.

5. Use of the compound according to claim 1, wherein the compound is selected from the group consisting of Alexa Fluor 680, Cy5.5, Quasar 705, ATTO 680, Alexa Fluor 700 and Tye 705.

6. Use of the compound according to claim 1 for performing multiplex PCR on non-droplet based dPCR systems, nucleic acid amplification with multiplex reactions or multiplex PCR, wherein the PCR is selected from the group consisting of digital PCR (dPCR), quantitative PCR (qPCR), reverse transcription PCR (RT-PCR), reverse transcription quantitative PCR (RT-qPCR) and real-time PCR.

7. Use of the compound according to claim 1, wherein the compound is bound to a probe or primer as fluorescent label and is used for an application selected from the group consisting of absolute quantification of nucleic acids in a sample, mutation detection in nucleic acids, analysis of genome editing, analysis of copy number variation and analysis of gene expression.

8. A method for performing multiplex PCR on non-droplet based dPCR systems, nucleic acid amplification with multiplex reactions or multiplex PCR, wherein the PCR is selected from the group consisting of digital PCR (dPCR), quantitative PCR (qPCR), reverse transcription PCR (RT-PCR), reverse transcription quantitative PCR (RT-qPCR) and real-time PCR, wherein a compound according to any one of the preceding claims is added to the PCR reaction as reference dye or dye for target detection, wherein the compound is bound to a probe or primer as fluorescent label.

9. The method of claim 8, which is performed for an application selected from the group consisting of absolute quantification of nucleic acids in a sample, mutation detection in nucleic acids, analysis of genome editing, analysis of copy number variation and analysis of gene expression.

10. A method for multiplex detection of biological or chemical components in a sample, wherein a compound according to claim 1 is added as reference dye or dye for target detection.

11. Composition comprising:
a) at least one fluorescent dye with a high Stokes shift according to claim 1
b) a heat-resistant DNA polymerase,
c) mixture of dNTPS,
d) a PCR buffer solution and
e) bivalent ions, preferably Mg²⁺ ions.

12. Use of the composition of claim 11 as a master mix.

13. A kit for performing multiplex PCR reactions comprising
a) the composition of claim 11 and,
b) a manual.
